(19) United States
(12) Patent Application Publication
(10) Pub. No.: US 2020/0268565 A1
(43) Pub. Date: Aug. 27, 2020

(54) ABSORBENT ARTICLE PROVIDED WITH A QR-CODE

(71) Applicant: Essity Hygiene and Health Aktiebolag, GÖTEBORG (SE)

(72) Inventors: Wen Shiuan Ling, Shah Alam, Selangor (MY); Chow Peng Ong, Klang Selangor (MY); Huey Chyi U, Kuala Lumpur (MY)

(73) Assignee: Essity Hygiene and Health Aktiebolag, GÖTEBORG (SE)

(21) Appl. No.: 16/643,741

(22) PCT Filed: Sep. 7, 2017
(86) PCT No.: PCT/SE2017/050886

§ 371 Date: Mar. 2, 2020

(51) Int. Cl. A61F 13/493; A61F 13/514; A61F 13/511; A61F 13/84; A61F 13/49

(52) U.S. Cl. CPC A61F 13/493; A61F 13/51496; A61F 13/5116; A61F 2013/51165; A61F 13/4906; A61F 2013/8497; A61F 13/84

(57) ABSTRACT

A disposable absorbent article such as a diaper, pant diaper, a sanitary pant or incontinence garment, intended to be worn around the waist of a wearer is provided. The absorbent article has a chassis having a front portion, a back portion and a crotch portion therebetween. The absorbent article optionally has one or more fastening tabs attached to the back or front portion of the chassis. The absorbent article has an absorbent core secured to the chassis in at least the crotch portion. The absorbent article is provided with a QR-code on a garment facing-side thereof and on a carrier material in or on at least one of the front portion, back portion and/or on the one or more fastening tabs. The carrier material has a Gurley stiffness of 8 mgf or more.

### CROSS REFERENCE TO RELATED APPLICATIONS

This is a U.S. National Phase application of PCT/SE2017/050886, filed Sep. 7, 2017, the disclosure of which is incorporated herein by reference in its entirety for all purposes.

### TECHNICAL FIELD

The present invention relates to a disposable absorbent article, such as a diaper, pant diaper, a sanitary pant or incontinence garment, provided with a QR-code.

### BACKGROUND

Absorbent article, of the kind to which this disclosure relates are worn against the skin and comprise a topsheet, an absorbent core and a backsheet layer. An additional layer of material is often applied to the garment-facing side of the backsheet as an external cover. The external cover of the absorbent article can be formed from a non-woven material to provide a soft texture to the absorbent article. The external cover may also form a landing zone for mechanical fasteners, and may therefore include mechanical fastener receiving elements, such as loops. Mechanical fastening elements are also often provided on fastening tabs attached to the front or rear portion of the chassis.

The external cover of the absorbent article may also include printed images to improve the aesthetic appeal of the absorbent article. A conventional approach to provide such aesthetic appeal includes printing images on the exterior, garment-facing, surface of the external cover. This normally provides good image resolution. On baby diapers such printed images may be in the form of cute animals or other images making the diapers more underwear like for them to be worn alone by them self as a pant. The diapers may also be provided with images for brand-identification of the diapers. Such images are printed in clearly distinguishable colors and sized to be visible to the naked eye.

However, there may also exists a desire to provide absorbent article with images which are supposed to be read, not only by the naked eye, but by other means with higher requirements with regard to precision of the readability. For such articles it has been found that there exists a need to ensure the readability of the images.

### SUMMARY

The above and other objects may be provided by an absorbent article according to claim 1. Further embodiments are set out in the dependent claims, in the following description and in the drawings.

As such, the present disclosure relates to a disposable absorbent article such as a diaper, pant diaper, a sanitary pant or incontinence garment, intended to be worn around the waist of a wearer. The absorbent article comprises a chassis having a front portion, a back portion and a crotch portion there between. The absorbent article furthermore optionally comprises one or more fastening tabs attached to the back or front portion of the chassis. The absorbent article has a wearer-facing side and a garment-facing side. The absorbent article further comprises an absorbent core secured to the chassis in at least the crotch portion. The absorbent article is provided with a QR-code on a garment facing-side thereof and on a carrier material in or on at least one of the front portion, back portion and/or on the one or more fastening tabs. The carrier material has a Gurley stiffness of 8 milligrams of force (mgf) or more, as measured according to the Gurley Stiffness test method TAPPI T 543 om-05, Bending resistance of paper (Gurley-type tester).

The bending resistance is measured according to a well-known standard in the paper and nonwoven industries—Tappi T 543 om-05, Bending resistance of paper (Gurley-type tester). A hanging strip of the sheet material presses against a vane attached to a pendulum, and the amount of deflection in the pendulum indicates the bending resistance. The result is presented in milligram force or in Gurley units (the two units are identical). It is possible to perform the test on different sample sizes, according to procedures and calculations laid out in the standard. Ten samples are cut from the material under investigation, five of them represent the material as seen in the diapers longitudinal direction, and five represent the cross direction. The average from the ten samples equals the bending resistance in the context of this invention. All testing is made in a laboratory environment of 23° C. and 50% relative humidity.

The two averages (longitudinal direction and cross direction) are reported separately, and in the context of this disclosure both values must fulfil the stipulated Gurley stiffness values. When measuring the Gurley stiffness of the carrier material, the carrier material provided with QR-code should be cut out, including the QR-code, from the absorbent article according to the test method, if the carrier material is a laminate, the measurements should be performed on the laminate.

The fact that the QR-code is provided on a carrier material having a Gurley stiffness of 8 mgf or more, as measured by the Gurley Stiffness tester, significantly improves the reading of the QR-code. The relatively high material stiffness also provides for an enhanced repeatability of the QR-code reading and it improves the readability over time. The Gurley stiffness of the material according to the present disclosure provides for that the carrier material is resistant to wrinkles etc. resulting from the use of the absorbent article which may otherwise negatively affect the readability of the QR-code.

Optionally, the carrier material has a Gurley stiffness of 10 mgf or more, as measured by the Gurley Stiffness tester. The carrier material may have a Gurley stiffness of from 10 mgf to 1000 mgf, such as from 12 mgf to 1000 mgf, such as from 10 mgf to 500 mgf, such from 12 mgf to 500 mgf. The Gurley stiffness may also be not more than 200 mgf or not more than 100 mgf. Optionally, the carrier material has a uniform stiffness, at least over the area provided with the QR-code.

The QR-code may be provided on the absorbent article to enable reading by means of a smartphone linking to on online platform, such as for displaying bed time stories and/or the story of the day, which parents can read for their children before bedtime or after/before changing the diaper. For example, the consumer may after scanning of the QR-code be directed to a landing page with access to the stories upon selection of the age of the baby/child. Changing the diaper of a baby may be connected to discomfort for the baby and struggling to keep the baby still while positioning the diaper. If the baby instead connects the diaper change with story time this may turn into a positive moment. Additionally, this would enable parents to always bring along bed time stories and other stories without having to remember to bring books along. The QR-codes may of course also be used for other purposes, such as for loyalty programs.

Optionally, the QR-code is a printed QR-code. The printing may for example be a flexographic print, gravure printing or an embossed print.

A printed QR-code gives the impression that the QR-code is an integrated part of the absorbent article and printing is also an easy way to provide the absorbent articles with the QR-code. It has been found by the present inventors that printing of the QR-code on a relatively stiff material increases the readability of the QR-code. The provision of a QR-code on a too flexible material will negatively impact the readability as creases or unwanted stretching of the carrier material may occur and interfere with the readability of the QR-code.

Optionally, the carrier material is any one of a nonwoven material, a knitted material, a woven material or a film material or a combination, such as a laminate, of said materials.

The fact that the carrier material is any one of; a nonwoven material, a knitted material, a woven material or a film material or a combination, such as a laminate, of said materials, which are commonly used materials in absorbent articles, preserves the appearance and function of the absorbent article, while adding the functionality of the QR-code.

Optionally, when the carrier comprises or consists of a nonwoven material, the nonwoven material comprises at least two or more layers of spunbond and/or meltblown fibers. Such nonwoven material have been found advantageous as carrier materials due to the possibility to provide a relatively stiff and smooth carrier surface for the QR-code, while preserving the comfort and appearance of the absorbent article when applied to a user.

Optionally, the spunbond and/or meltblown fibers of the layers may have a diameter of from 1.5 to 2.5 microns. The fact that the spunbond and/or meltblown fibers, provided on a garment-facing/printing side of the carrier material, have such fine diameter size may enhance the smoothness of the carrier material surface and thereby enhance the readability of the QR-code, compared to if the carrier material surface is irregular.

Optionally, the carrier material has a basis weight from 50 gsm to 150 gsm.

The fact that the carrier material has a basis weight of at least 50 gsm reduces the risk of creasing and folding of the carrier material, which may negatively impact the readability of the QR-code. A basis weight of at least 50 will thereby aid in ensuring a consistent readability of the QR-code provided on the absorbent article, even after a certain time of use and activity of the wearer.

Optionally, the absorbent article is provided with two or more QR-codes on a garment facing-side thereof, each of the QR-codes being provided on one or more carrier material(s) in or on at least one of the front portion, back portion and/or on the one or more fastening tabs. Optionally, the absorbent article is provided with three or more QR-codes on a garment facing-side thereof, each of the QR-codes being provided on one or more carrier material(s) in or on at least one of the front portion, back portion and/or on the one or more fastening tabs.

Optionally, the absorbent article comprises at least one fastening tab, and the carrier material constitute, or form part of, the at least one fastening tab. The absorbent article may comprise at least two fastening tabs, attached, directly or indirectly, to a respective longitudinal side edge of the back or front portion of the chassis. Optionally, the fastening tabs are provided with one or more QR-codes on each fastening tabs. The fastening tabs may also be provided with two or more QR-codes on each fastening tabs, such as three or more QR-codes on each fastening tabs.

By “fastening tab” herein is meant a piece of material comprising a fastening component adapted to be fastened to a receiving component on the absorbent article. The fastening tab may be a square or rectangular piece of material, directly or indirectly, attached to the longitudinal side edge(s) of the chassis back or front portion. The fastening tab may also include a larger shaped piece of material, directly or indirectly, attached to the longitudinal side edge(s) of the chassis back or front portion. Such larger/shaped fastening tab(s) may function as side panel(s) as well as fastening components) for the absorbent article. The fastening component may be any conventionally used fastening components for absorbent articles, such as for example, an adhesive or a hook material.

The fastening tab may comprise a material with a higher stiffness, without decreasing the soft and underwear-like perception of the absorbent article. The fastening tabs also constitute clearly visible parts of the absorbent article when in use, as these are often attached on the front portion of the chassis and on the stomach of the user/baby. The fastening tabs are thus provided on the side normally facing the caregiver/parent during interaction between the baby and parent. This also allows the baby to take part in the scanning of the QR-code, which may increase the interest in the story appearing upon linkage to the website.

The fact that the QR-code(s) has been printed on the absorbent article fastening tab thus provides the QR-code(s) on a material which may be a stiff material without influencing the comfort of the absorbent article by including a stiffer material for example as a backsheet material.

Optionally, the QR-code(s) is/are provided on an outermost layer, facing away from the user, and with no overlaying layers. This improves the readability after the absorbent article has been put on the wearer, such as on the baby/child. It has been found that also an outer thinner layer, such as a thin nonwoven layer, provided on top of the carrier material may have an impact on the readability.

Optionally, the QR-code(s) has/have a size of at least 10×10 mm, such as from 10×10 mm to 60×60 mm, such as from 10×10 mm to 30×30 mm. This provides for a proper readability of the QR-code(s), while maintaining an aesthetical appearance for the absorbent article, such as for a baby diaper.

Optionally, the carrier material has an extensibility of no more than 4%, as measured upon application of a load of 100 g per centimetre of the material width. The extensibility of the carrier material may be no more than 4% in both the length and the width direction.

The extensibility of the material according to the disclosure herein is measured on material strips, cut such that their length dimension corresponds to the diaper's cross direction. Two lines should be drawn across the strip, and an initial length between them measured. Then one end of the strip should be attached into a fixed clamp, and a force corresponding to 100 grams per width centimeter of the strip applied via a second, moveable clamp. This stress should be held for ten seconds, and then the (extended) length between the lines should be measured. The percent elongation is then determined via this formula: ((extended length−initial length)/initial length)×100. All testing being made in a laboratory environment of 23° C. and 50% relative humidity.

The fact that the carrier material has an extensibility of no more than 4%, prevents unwanted stretching of the material and thus decreased readability of the QR-code. Stretching of the material decreases readability of the QR-code as the pattern of the black/coloured squares in the squared QR-code grid may be affected by the stretching.

Optionally, the carrier material is non-extendible.

Optionally, the QR-code has a colour difference of about 10 to about 90 in respect to said carrier material. The colour difference of the QR-code in respect to the carrier material shows the degree of the colour difference (or distance) of the primary colour of the QR-core from standard white colour. This may enhance the readability. It may be desired that absorbent article is provided with graphic(s) on the outer side of the chassis, and that the QR-code is provided in the same colour or within the same “colour system” as the graphic(s).

The colour difference is measured in accordance with CIE 1976 L*a*b*: CIE Publication 15.2 (1986) by using the white standard plate which has the standard white color (L*=93.95, a*=0.26, b*=4.95). The measurements should be performed by using a light source “D50 (corresponding to daylight and a correlated colour temperature of about 5000K)” at a viewing angle of 10° (degrees). Before the measurement, ten carrier material colored in the color of the QR-code to be measured are piled up to form a sample material. In the measurement, the white standard plate is placed on the flat table. The sample material (i.e., the ten piled sheets) is put on the white standard plate in a flat condition. The sample material is illuminated by the light source with an incident angle of 45° . The reflection light which is reflected from the sample material is received by the photo detector with a receiving angle of 0°. The reflection light is detected by the photo detector. The detected reflection light is converted into the luminosity (L*1), the red-green chromaticity (a*1), and the yellow-blue chromaticity (b*1) by the colorimeter.

After the sample carrier material is replaced with the standard white surface, the above measurement is conducted for the standard white surface. The detected reflection light by the photo detector is converted into the luminosity (L*w), the red-green chromaticity (a*w), and the yellow-blue chromaticity (b*w) by the colorimeter. The above process is repeated for each carrier material at least five times and the average values L*1, a*1, b*1, L*w, a*w, and b*w are calculated and recorded by the colorimeter. Thus, two sets of photo data (L*1, a*1, b*1) and (L*w, a*w, b*w) are obtained. The total color difference value (A*E) is then obtained by the following formulas: ΔL*=L*1−L*w, Δa*=a*1−a*w, Δb*=b*1−b*w and ΔE*=√((ΔL*)²+(Δa*)²+(Δb*)². These data are calculated and recorded by the colorimeter.

With “colour system” herein is meant a group of colours which are different but similar to one another. Colour systems include a blue colour system, a red colour system, a pink colour system, an orange colour system, a yellow colour system, and a green colour system. Colour systems can be defined by the International Commission on Illumination (CIE), i.e., CIE L*a*b hue angle. For example, “blue colour system” is defined as CIE L*a*b hue angle ranging from −40 degrees to −145 degrees, “pink colour system” is defined as CIE L*a*b hue angle ranging from −40 degrees to 20 degrees, “orange colour system” is defined as CIE L*a*b hue angle ranging from 20 degrees to 55 degrees, “yellow colour system” is defined as CIE L*a*b hue angle ranging from 55 degrees to 110 degrees, and “green colour system” is defined as CIE L*a*b hue angle ranging from 110 degrees to −145 degrees.

With “graphic” herein is meant a pattern that is constituted by a shape(s), a figure(s), a symbol(s) or character(s).

The fact that the absorbent article is provided with graphic(s), and that the QR-code is provided in the same colour or within the same colour system as the graphics gives the absorbent article an underwear-like appearance as the QR-code becomes more discreet and an integrated part of the absorbent article graphic(s). The disposable absorbent article may comprise a print having a distinctive colour and a QR-code having the same distinctive colour.

Optionally, the carrier material has an opacity of about 80% to about 100%.

The opacity of the carrier material shows the degree of transparency or translucency. When a carrier material has an opacity of 0%, the carrier material is completely transparent. On the other hand, if the carrier material has an opacity of 100%, the carrier material has no transparency, i.e., no light is transmitted through the material. The high opacity of the carrier material may support the visibility and the readability of the QR-code as the contrast between the carrier material and the QR-code is enhanced with high-opacity materials, such as when the carrier material is a white, uncolored, high opacity material.

If the carrier material is, or forms part of, a fastening tab, the high opacity may be of importance to prevent underlying graphics, when the absorbent article is in a closed and fastened form, to obscure the readability of the QR-code.

### BRIEF DESCRIPTION OF THE DRAWINGS

The present invention will be further explained hereinafter by means of non-limiting examples and with reference to the appended drawings wherein:

FIG. 1 illustrates a diaper according to the present disclosure, with QR-codes provided on the fastening tabs; and

FIG. 2 illustrates a fastening tab according to FIG. 1.

### DETAILED DESCRIPTION OF PARTICULAR EMBODIMENTS

It is to be understood that the drawings are schematic and that individual components are not necessarily drawn to scale. The diaper and the fastening tabs shown in the figures are provided as examples only and should not be considered limiting to the invention. Accordingly, the scope of invention is determined solely by the scope of the appended claims.

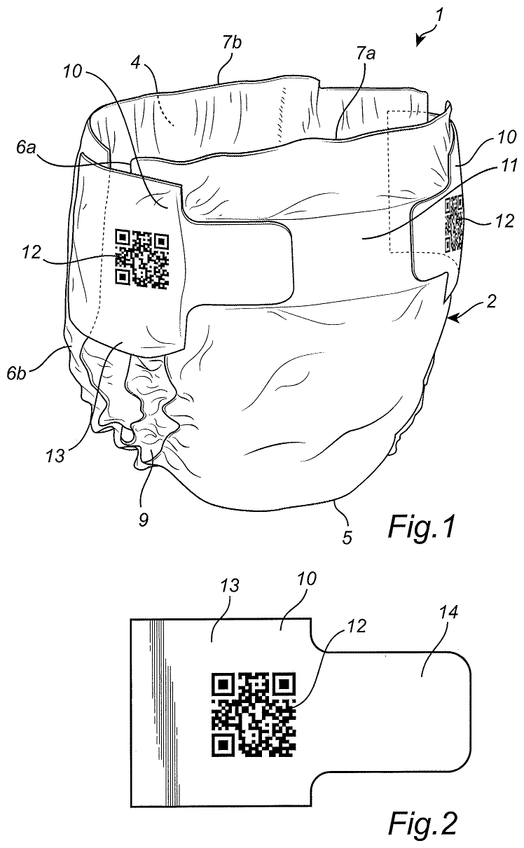

FIG. 1 illustrates an absorbent article 1, in the form of a diaper, in a closed configuration. The absorbent article 1 comprises a chassis 2 having a front portion 3, a back portion 4 and a crotch portion 5 there between. The absorbent article furthermore comprises a front waist 5 edge 7a and a back waist edge 7b and first and second longitudinal side edges 6a,6b extending from the front and back waist edges 7a,7b. The absorbent article also comprises leg elastic 9 extending along the longitudinal side edges 6a,6b in the crotch portion 5. The absorbent article 1 according to FIG. 1 furthermore comprises one fastening tab 10 extending from the first longitudinal side edge 6a, and one fastening tab 10 extending from the second longitudinal side edge 6b. The fastening tabs 10 are attached directly to the back portion 4 of the chassis 2. The fastening tabs 10 are in the form of large shaped and non-rectangular carrier materials, each comprising broader sections 13 attached at the longitudinal side edges 6a,6b of the back portion 4 of the chassis 2, and more narrow fastening sections 14. The fastening sections 14 comprise fastening means on a wearer facing side thereof. The fastening means may for example be adhesive or hooks. The absorbent article 1 furthermore comprises a receiving component 11 provided on an outer surface of the front portion 3 of the chassis 2. The absorbent article 1 is provided with two QR-codes 12 arranged on the outer surface of the fastening tabs 10. However, the absorbent article may also be provided with more than one QR-code on each carrier material, such as two, three or four QR-codes on each carrier material. The QR-codes may be identical or different QR-codes. The fastening tabs 10 are composed of a nonwoven material having a stiffness of 16 mgf as measured by the Gurley stiffness tester and the TAPPI test method T 543 om-05, Bending resistance of paper (Gurley-type tester).

FIG. 2 shows the fastening tab 10 from FIG. 1. In this figure, the QR-code 12 is printed on the fastening tab 10, but slightly off set the area comprising the fastening component on the wearer facing side.

Table 1, shown below, shows test data from measurements with respect to the Gurley stiffness, as measured according to the TAPPI specification T 543 om-05 by the Gurley Stiffness tester. In the test, measurements were performed on a fastening tab according to the present disclosure comprising a 55 gsm SSMMS nonwoven, from Fibertex and compared to a breathable backsheet laminate comprising a 10 gsm polypropylene/polyethylene film laminated with a 12 gsm polypropylene nonwoven.

Measurements in respect to the extensibility of different types of material were also made, comparing the fastening tab nonwoven with the backsheet laminate and an elastic side panel. The extensibility measurements were performed by removing, by cutting, the materials in the lateral direction of the diaper, and by applying a load of 100 grams per centimetre of the material width. The elastic side panel is taken from the commercially available “Libero Touch” from SCA Hygiene Products AB. The elastic side panel was a laminated material, comprising an elastic film in the middle (40 gsm, styrenic block copolymer) glued to two outer, covering nonwovens (27 gsm each, carded polypropylene fibers).

TABLE 1

| | Gurley | |
|---|---|---|
| | stiffness | |
| | (mgf) | Extensibility |
| Nonwoven-Fastening tab | 16 | 3% |
| Backsheet laminate | 2 | 5% |
| Elastic side panel | | 70% |

As illustrated by the measurements presented in table 1, the difference in stiffness is significant between the fastening tab and the backsheet laminate, corresponding to a conventional backsheet laminate for a baby diaper. The extensibility of the nonwoven used as fastening tab and fulfilling the Gurley stiffness requirements are also very low, 3%, as shown in the third column in table 1.

Table 2, shown below, shows test data from measurements with regards to the readability for a fastening tab according to the present disclosure and for a 13 gsm backsheet laminate comprising a polypropylene film and a SMMS/SSMMS nonwoven (Fibertex Personal Care Sdn Bhd, spec. No. CT-MT-SPFB08), and a fastening tab. The backsheet laminate comprises a polyethylene film and a nonwoven, wherein the QR-code is printed on the polyethylene film and the fastening tab comprises a SSMMS/SSMMS nonwoven. The QR-codes were printed with flexographic print and the ink used was colour P455, L:70, a: −1.95, b: 12.00, max Delta E: 4 and density: 0.40-0.45. The results are indicated with either a 0 for a successful read and 1 for a failed read. The tests were performed under the same conditions and the scanning of the QR-code was performed with a mobile phone Samsung Galaxy Note II.

The procedure comprised the following steps;
1) Take one roll of the printed side panel/fastening tab nonwoven or alternatively one roll of the printed backsheet laminate and cut into samples having a width of 141 mm and a sample length of 3750 mm
2) ensure that there are no stains, folds or crumple mark on the QR-code printed area,
3) Take the mobile device, open the WeChat app on the mobile device. Tap the discovery button, and then select the Scan QR code option,
4) set up a mobile phone holder on a table,
5) place the mobile phone in the mobile phone holder such that the mobile phone camera is directed towards the table at a distance of 90 mm,
6) place a table lamp next to the mobile phone holder and switch on the lamp,
7) use a 0 for pass reading and a 1 as fail reading.

TABLE 2

| Backsheet | Fastening | Fastening |
|---|---|---|
| laminate | tab R | tab L |
| 1 | 0 | 0 |
| 0 | 0 | 0 |
| 1 | 0 | 0 |
| 1 | 0 | 0 |
| 1 | 0 | 0 |
| 0 | 0 | 0 |
| 1 | 0 | 0 |
| 1 | 0 | 0 |
| 1 | 0 | 0 |
| 0 | 0 | 0 |
| 0 | 0 | 0 |
| 1 | 0 | 0 |
| 1 | 0 | 0 |
| 0 | 0 | 0 |
| 1 | 0 | 0 |
| 0 | 0 | 0 |
| 1 | 0 | 0 |
| 1 | 0 | 0 |
| 1 | 0 | 0 |

As illustrated in table 2 above, a consistent readability is provided when printing the QR-code on the fastening tab having a stiffness of 16 gsm as measured by the Gurley Stiffness tester, compared with when the printing is provided on the backsheet of the absorbent article.

1-14. (canceled)

15. A disposable absorbent article such as a diaper, pant diaper, a sanitary pant or incontinence garment, intended to be worn around the waist of a wearer, said article comprising a chassis having a front portion, a back portion and a crotch portion there between, optionally one or more fastening tabs attached to said back portion and/or to said front portion of said chassis, said absorbent article having a wearer-facing side and a garment-facing side, said article further comprising an absorbent core secured to the chassis in at least the crotch portion, wherein a QR-code is provided on a garment-facing side and on a carrier material in or on at least one of said front portion, back portion and/or said one or more fastening tabs, and that said carrier material has a Gurley stiffness of 8 mgf or more, as measured according to the Gurley Stiffness test method TAPPI T 543 om-05, Bending resistance of paper (Gurley-type tester).

16. The disposable absorbent article according to claim 15, wherein said carrier material has a Gurley stiffness of from 10 mgf to 1000 mgf.

17. The disposable absorbent article according to claim 15, wherein said QR-code is a printed QR-code.

18. The absorbent article according to claim 15, wherein said carrier material is selected from the group consisting of a nonwoven material, a knitted material, a woven material, a film material, and a laminate of at least two thereof.

19. The absorbent article according to claim 18, wherein said carrier comprises a nonwoven material comprising at least two or more layers of spunbond and/or meltblown fibers.

20. The disposable absorbent article according to claim 19, wherein said fibers of said layers have a diameter of from 1.5 to 2.5 microns.

21. The disposable absorbent article according to claim 15, wherein said carrier material has a basis weight of from 50 gsm to 150 gsm.

22. The disposable absorbent article according to claim 15, wherein said absorbent article comprises at least one fastening tab, and wherein said carrier material forms part of said at least one fastening tab.

23. The disposable absorbent article according to claim 15, wherein said QR-code is provided at an outermost layer, facing away from the user.

24. The disposable absorbent article according to claim 15, wherein said carrier material has an extensibility of no more than 4%, as measured upon application of a load of 100 g per centimetre of the material width.

25. The disposable absorbent article according to claim 15, wherein said carrier material is non-extendible.

26. The disposable absorbent article according to claim 15, wherein said carrier material has an opacity of about 80% to about 100%.

27. The disposable absorbent article according to claim 15, wherein said disposable absorbent article comprises a print having a colour and wherein said QR-code has the same colour.

28. The disposable absorbent article according to claim 15, wherein said disposable absorbent article is provided with two or more QR-codes.
